(19) United States
(12) Patent Application Publication
(10) Pub. No.: US 2011/0105819 A1
(43) Pub. Date: May 5, 2011

(54) ZEOLITE CATALYST ZEOLITE SECONDARY STRUCTURE

(76) Inventors: Petr Vasiliev, Taby (SE); Lennart Bergström, Enskede (SE); Niklas Hedin, Norrtalje (SE)

(21) Appl. No.: 12/936,152

(22) PCT Filed: Apr. 1, 2009
(86) PCT No.: PCT/SE2009/050343

§ 371 Date: Jan. 3, 2011

(30) Foreign Application Priority Data
Apr. 4, 2008 (SE) 0800765-0

(51) Int. Cl. C07C 5/22; C01B 39/02; B01J 29/06

(52) U.S. Cl. 585/481; 423/700; 502/60

(57) ABSTRACT

A zeolite secondary structure essentially free from binders and formed from zeolite powder (primary particles), wherein the tensile strength of the secondary structure is at least about 0.4 MPa. The use of the zeolite secondary structure materials as catalyst in hydrocarbon conversion processes.

### FIELD OF INVENTION

The present invention refers to a zeolite secondary structure comprising less than about 10% by weight of binders and the use of the zeolite secondary structure as a catalyst for hydrocarbon conversion processes.

### BACKGROUND OF THE INVENTION

Different types of zeolites are widely used in industry as e.g. adsorbents, and catalysts, particularly for e.g. gasoline upgrading processes.

The size of zeolite particles typically in the rage of from 0.5 to 20 μm is often too small to be convenient for practical applications. Many catalysts and adsorbent applications require that zeolite particles, in the form of e.g. powders and herein referred to as primary particles, can be produced in macroscopic form, herein referred to as secondary structures. Examples of suitable forms for the zeolite secondary structures are granules, pellets, cylinders and discs. Such secondary structures can be produced by extruding a zeolite powder body followed by a heat treatment or by pressing a powder body into a pellet followed by a heat treatment. For example, fixed bed catalysts of cylindrical shape generally range from about 3 to 50 mm in diameter and have length-to-diameter ratios of about 1 for pelletised catalysts and up to about 3 or 4 for extrudates. Pellets or extrudates smaller than about 1-2 mm in diameter may cause excessive pressure drop through the bed. In extrusion processes, the zeolite crystals are extruded together with a non-zeolitic binder and an extrudate secondary structure is obtained after drying and calcination. The non-zeolitic binders are usually added to impart a high mechanical strength and resistance to attrition of the extrudate secondary structure. Examples of suitable binders include materials such as alumina, silica, and various types of clays.

Although zeolite secondary structures that contain non-zeolitic binders have much higher strength and attrition resistance than zeolite secondary structures that have been produced by traditional processes without the presence of any binders, the performance of the resulting catalyst is often reduced because of the binder. The binder can result in a reduction of effective surface area of the catalyst and reduce the activity. The binder can also introduce diffusional limitations and slow down the rate of mass transfer to and from the pores of the zeolite secondary structure which can reduce the effectiveness of the catalyst. Furthermore, the binder may participate in the reactions itself or affect the reactions that are catalyzed by the zeolite, e.g. in hydrocarbon conversion reactions, such that undesirable products are formed. Accordingly, it is desirable that zeolite catalysts, e.g. used in hydrocarbon conversion, contain a minimum amount of non-zeolitic binders.

U.S. Pat. No. 6,977,320 B2 discloses a zeolite bound zeolite catalyst comprising first crystals of a first zeolite and a binder comprising second crystals of a second zeolite. The second zeolite crystals bind the first zeolite crystals by adhering to the surface of the first zeolite crystals thereby forming a secondary structure. Preferably, the second zeolite crystals bind to the first zeolite crystals by intergrowing. The hydrothermally produced zeolite catalyst is preferably substantially free from non-zeolitic binder.

U.S. Pat. No. 5,098,894 relates to a binderless zeolite of MFI type, i.e. TSZ and ZSM-5. Macroscopic structures of TSZ or ZSM-5 are formed by molding a mixture of TSZ and a silica/alumina binder into pellets and subjecting the pellets to a hydrothermal treatment whereby a binderless zeolite is obtained.

Japanese published application Kokai No 11(1999)-228238 discloses a process for obtaining a crystalline porous structure comprising molding a crystalline microporous powder not containing molding and sintering aids using spark plasma sintering. The sintering is conducted at temperatures ranging from 100° C. to 800° C.

One objective with the present invention is to provide a zeolite secondary structure having a sufficient mechanical strength while not significantly deteriorating the performance, such as catalytic performance, compared to the performance of the primary zeolite particles. Another objective is to provide a zeolite secondary structure essentially free from binders (such as non-zeolitic binders) having a sufficient mechanical strength. Yet a further objective is to provide a zeolite secondary structure essentially free from binders having sufficient mechanical strength for the conversion of hydrocarbons, in particular isomerisation of xylene, without significantly decreasing the performance with respect to conversion and/or selectivity.

### BRIEF DESCRIPTION OF FIGURES

FIG. 1 Mechanically stable, multiporous pellets prepared by rapid heating of an assembly of ZSM-5 zeolite primary particles in dies of cylindrical shape with a different height/diameter ratio

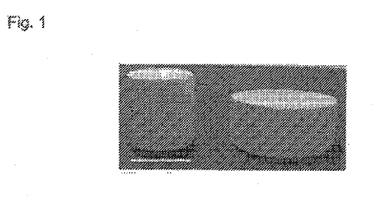

### SUMMARY OF THE INVENTION

The present invention is directed to a zeolite secondary structure which comprises less than about 10% by weight of binders and having a tensile strength of at least about 0.40 MPa. The strength of the secondary structure is obtained by a process comprising providing zeolite primary particles, usually in powder form, rapid heating the primary particles to above about 800° C. at a heating rate of at least about 10° C. per minute under a pressure of at least about 5.0 MPa. The zeolite secondary structure is preferably used as a catalyst in various hydrocarbon conversion processes including cracking, alkylation, dealkylation, disproportionation, transalkylation, dehydrogenation, hydrocracking, isomerisation, dewaxing, oligomerisation and reforming.

### DETAILED DESCRIPTION OF THE INVENTION

The present invention relates to a zeolite secondary structure comprising less than about 10% by weight of binders formed from zeolite primary particles, where the tensile strength of the secondary structure is about 0.40 MPa. Many zeolites are not found in nature and are synthetically products. Such synthetically farmed zeolites are particles typically in the range between about 0.5 μm up to about 20 μm, and referred to herein as primary particles. Of course, primary particles also encompass naturally occurring zeolites in the size range mentioned above. For many purposes zeolite primary particles are not appropriate, e.g. due to a high pressure drop. Thus, zeolite primary particles are often transformed into secondary structures of macroscopic form. Zeolite secondary structures can have various forms and are significantly larger than the primary particles usually an average size above about 1 mm. The form of the secondary structure is dependent on the use including but not limited to granules, pellets, cylinder forms, and discs. Zeolite secondary structures used as catalysts in fixed bed reactors can have varying forms including rings, balls and complex forms. Cylindrically formed secondary structures used for fixed bed reactors may have a diameter of from about 3 to 50 mm and a length to diameter ratio of about 1 up to about 5.

As used herein, zeolitic materials are microporous crystalline aluminosilicates zeolitic materials can be distinguished from dense tectosilicates by referring to the framework density (FD), i.e. the number of tetrahedrally coordinated atoms (T-atoms) per 1000 Å³ as disclosed in the “Atlas of Zeolite Framework Types”, Baerlocher, Meier, Olson, Fifth Ed. Aluminosilicates having a framework density (FD) above about 21 T-atoms per 1000 Å³ have dense tetrahedral frameworks whereas the crystalline microporous aluminosilicate materials of the present invention have a framework density FD of up to about 21 T-atoms per 1000 Å³. Accordingly, as used herein zeolite refers to crystalline microporous aluminosilicates having a FD of up to about 21 T-atoms per 1000 Å³, suitably the FD is from about 12 up to about 21 T-atoms per 1000 Å³. Further, other atoms being tetrahedrally coordinated may be present in the zeolite crystal structure including but not limited to Ga, Ge, B, Be-atoms. The zeolite secondary structure may be aluminosilicates having at least about 90% by weight of the aluminosilicate in crystalline form. Suitably, the crystalline aluminosilicate is in a hydrogen form and/for as a salt with metal ions. Further, the zeolite framework may present defects such as non-bridging oxygen, vacant cites, mesopores; and the coordination of the T-atoms may be modified by species present in the micropores.

Zeolite secondary structures are desirable in many applications. Zeolite secondary structures are commonly obtained by the addition of a non-zeolitic binder material prior to formation of the secondary structure. The non-zeolitic binder confers to the secondary structure inter aria mechanical strength and resistance to attrition. However, the improved strength and attrition resistance by the use of non-zeolitic binders when forming secondary zeolite structures are usually offset by inter alia a reduction of performance. Commonly used non-zeolitic binders are various amorphous materials like aluminia, silica, titanic, and various types of clays. The present zeolite secondary structure comprises less than 10% by weight of binders, based on total zeolite material excluding binder/binders. By binder or binders is herein meant any non-zeolitic material. Preferably, the zeolite secondary structure comprises less than about 5% by weight of binders, suitably less than about 1% by weight. According to one embodiment of the present invention the zeolite structure is essentially free from binders or even free from binders, i.e. binder-less. Free from binders implies herein that the amount of binders in the zeolite is below detection by powder x-ray diffraction.

According to the present invention a zeolite secondary structure is provided comprising less than about 10% by weight of binders and having high strength. Also, a high degree of attrition resistance is also ensured. As used herein the tensile strength is measured according to the diametral compression test, also known as the Brazilian test. The specimens are subjected to diametral compression using two parallel plates. Tensile strength is calculated as σ_{T}=2P/d·t·TT, where P=load at failure (N), d=specimen diameter (mm) and t=specimen thickness (mm). According to the present invention the tensile strength of the secondary zeolite structure is at least about 0.40 MPa, at least about 0.45 MPa, at least about 0.50 MPa, at least about 0.55 MPa, at least about 0.60 MPa suitably at least about 0.65 MPa, at least about 0.70 MPa, at least about 0.80 MPa, at least about 0.90 MPa, at least about 1.00 MPa. The tensile strength may be at least about 1.60 MPa, preferably at least about 2.00 MPa.

According to one embodiment of the present invention the crystallographic free diameter of the channels having most T-atoms of the zeolite secondary structure ranges of from about 0.3 nm up to about 1.3 nm. For the definition of “crystallographic free diameter reference is made to Atlas of Zeolite Framework Types”, Baerlocher, Meier, Olson, Fifth Ed. The zeolite secondary structure may have a pore size distribution with more than 25% of the pore volume in pores with radii from about 10 to about 10000 nm.

According to yet another embodiment of the present invention the zeolite secondary structure is obtained from primary zeolite particles of MR type, i.e. the framework type is MFI. Accordingly, the Zeolites of MFI type include e.g. ZSM-5, [As—Si—O]-MFI, [Ga—Si—O]-MR, AMS-1B, AZ-1, Bar-C, Boralite C, Encilite, FZ-1, LZ-105, Monoclinic H-ZSM-5, Mutinaite, NU-4, NU-5, Silicalite, TS-1, TSZ, TSZ-III, TZ-01, USC-4, USI-108, ZBH, ZKQ-1B, ZKQ-1B, and organic-free ZSM-5.

According to one embodiment the zeolite secondary structure is obtainable by a process comprising providing zeolite primary particles, heating the zeolite particles to a temperature of above about 800° C. at an average rate of at least about 10° C. per minute at a pressure of at least about 5.0 MPa whereby the secondary structure is formed. The starting temperature of the process may vary. As a matter of convenience, the starting temperature for the heating of the zeolite particles at a rate of at least 10° C. per minute is ambient temperature. The heating can be carried out at any pressure including vacuum, ambient pressure and elevated pressures and any pressures there between. Preferably, the heating is conducted under an elevated pressure, suitably at a pressure of at least about 5.0 MPa. Preferably, the pressure during heating is at least about 5.5 MPa, at least about 6.0 MPa, at least about 7.0 MPa, at least about 10.0 MPa, at least about 15.0 MPa, at least about 18.0 MPa, at least about 20.0 MPa. Typically, the pressure is between about 10 MPa up to about 40 MPa. By pressure is meant externally applied pressure. The heating rate is suitably at least about 20° C. per minute, at least about 30° C., at least about 40° C., preferably at least about 50° C. and preferably at least about 100° C. per minute. Improved results with respect to tensile strength are obtained if the zeolite is heated up to a temperature of about 900° C., up to about 940° C., and up to about 1000° C. Typically, the temperature should not exceed 1400° C. Higher temperatures than 1400° C. may significantly decrease the surface area of the secondary zeolite structure. Accordingly, the temperature may range from above about 800° C., such as from above about 820° C. up to about 1400° C., suitably the temperature is between about 850° C. to about 1300° C., between about 900° C. up to about 1250° C., between about 950° C. up to about 1200° C., between about 980° C. up to about 1150° C. Preferably, the temperature is maintained over a period of time after the maximum average temperature has been obtained prior to cooling. If the high (maximum) temperature is maintained for a period of time, the (high) temperatures refer to the average temperature during the period of time. Suitably, the average maximum temperature is maintained under a period of time ranging of less than about 60 minutes, suitably less than 15 minutes, preferably less than 5 minutes, such as between 0 sec. up to 5 min, suitably between 30 sec. up to 4 min. The temperature may fluctuate as long as the average temperature is above about or about the indicated maximum temperatures, e.g. 800° C. Typically, the high/maximum temperature may vary up to about 20%. The heating including in optionally maintaining the zeolite at the high temperature is followed by cooling. Suitably, this cooling is conducted at a cooling rate of at least about 1° C. per minute, preferably at a cooling rate of at least about 10° C. per minute. Typically, the zeolite is cooled down to ambient temperature. Preferably, the rapid heating process is conducted in a machine where the mass of the heated elements is relatively small to allow a rapid heating, and subsequently, rapid cooling process, more preferably, the process is conducted in a machine which consist of electrically conductive dies which can be heated by a pulsed current, and, most preferred, the electrically conductive dies are made of graphite. Preferably, the rapid heating process is conducted by simultaneously subjecting the zeolite powder (primary particles) assembly to a compressive pressure of more than 5 MPa, more preferably, at a compressive pressure between 10 and 40 MPa.

### Example 1

Binder-free ZSM-5 secondary structure formed by a rapid heating and cooling process.

1.5 g of the as-received ZSM-5 zeolite powder (primary particles) was loaded in cylindrical graphite dies, pre-compressed at room-temperature, and placed in a pulsed current processing machine (Dr. Sinter 2050, Sumitomo Coal Mining Co. LTD, Japan). The ZSM-5 particles were subjected to an uniaxial pressure of 20 MPa and heated to an average maximum temperature of 950° C., 1100° C. and 1200° C., respectively, in vacuum at an average heating rate of 100° C./min, with a holding time of 3 minutes at the maximum temperature. The powder assembly was cooled down quickly; it took less than 4 minutes to reach 200° C. The temperature was regulated using a feed-back regulator. The temperature was measured with a pyrometer that was focused on the surface of the graphite die.

The zeolite secondary structures, which also can be called pellets, produced with the process described above at a maximum temperature of 950° C. had a surface area, determined by five point BET analysis of nitrogen adsorption isotherms, of 350 m²/g and a pore volume of 0.59 cm³/g determined by mercury porosimetry and t-plot analysis of nitrogen adsorption isotherms. The zeolite secondary structure produced at a maximum temperature of 1100° C. had a surface area, determined by five point BET analysis of nitrogen adsorption isotherms, of 330 m²/g and a pore volume of 0.56 cm³/g, determined by mercury porosimetry and t-plot analysis of nitrogen adsorption isotherms.

The strength of the cylindrical zeolite secondary structures, determined by the diametral compression test, also known as the Brazilian test or splitting tensile test, were performed by applying a compressive load on the perimeter of the circular disc until a crack forms, causing failure of the specimen. Diametral compression test were carried out at ambient conditions using an electromechanical testing machine (Zwick Z050, Germany) at a constant cross-head displacement rate of 0.5 mm/min. The strength of the zeolite pellets were 2.4 MPa for the ZSM-5 pellet prepared by the process described above at a maximum temperature of 1200° C., 1.6 MPa for the ZSM-5 pellet prepared at a maximum temperature of 1100° C. and 0.7 MPa for the ZSM-5 pellet prepared at a maximum temperature of 950° C.

### Example 2

Xylene isomerisation results using the ZSM-5 secondary structures prepared according the process described in Example 1.

Zeolite powder (primary particles) and grinded zeolite pellets (secondary structures) were heated in a furnace at 500° C. for 6 hours, with a heating and cooling rate of 0.2° C./min to obtain the ion exchanged H⁺ form. A tubular fixed bed reactor of stainless steel was used for the catalysis experiment. The internal diameter of the reactor was 17 mm and the internal length is 200 mm. The zeolites were mixed with 90 wt % sea sand and ethanol and stirred until a homogenous mixture was obtained. The zeolite/sand mixture was subsequently loaded in the middle of the reactor, the beginning and end of the reactor was filled with glass beads.

Catalytic test were performed using p-xylene isomerisation reaction. The zeolites (primary particles and grinded secondary structures) were calcined in-situ at 450° C. for 6 h prior and in between testing. The feed was nitrogen saturated with p-xylene (>99%, Merck) at 60° C. and it was fed to the reactor. The feed and the products were analyzed with an online gas chromatograph (Varian CP 3800) with a polar column (CP Xylene) and a FID detector.

The result is given in table 1 and graph 1.

TABLE 1

| | Sample # | | | | | | | | |
|---|---|---|---|---|---|---|---|---|---|
| | 1 | 2 | 3 | 4 | 5 | 6 | 7 | 8 | 9 |
| Temperature | 950° C. | | | 1100° C. | | | Primary | | |
| | (square) | | | (rhombic) | | | particles | | |
| | | | | | | | (triangular) | | |
| Conversion % | 2.5 | 3.6 | 5.1 | 1.05 | 1.27 | 1.67 | 6.7 | 9.5 | 13.2 |
| m/o-xylene | 3.7 | 3.9 | 4.1 | 3.5 | 3.5 | 3.6 | 4.4 | 4.0 | 3.9 |
| ratio | | | | | | | | | |

Graph 1 shows the data of table 1.

The main products were o- and m-xylene. Samples 7-9 (primary particles) have the highest conversion of p-xylene from 6.5% to 13%. The secondary structures that have been prepared at 950° C. (sample 1-3) display a conversion between 2.5% and 5.1%. The secondary structures that have been prepared at 1100° C. (sample 4-6) display a conversion between 1.05% and 1.67%. The data in Graph 1 show that the zeolite secondary structures produced at both 950° C. and 1100° C. retain the m-xylene selectivity for the primary particles (equilibrium relationship is 2).

1. A zeolite secondary structure obtained from zeolite primary particles comprising less than about 10% by weight of binders, wherein the tensile strength of the secondary structure is at least about 0.40 MPa.

2. The zeolite secondary structure according to claim 1, wherein the tensile strength is at least about 0.45 MPa.

3. The zeolite secondary structure according to claim 1, wherein the secondary structure is obtained by a process comprising providing zeolite primary particles, heating the zeolite primary particles to above about 800° C. at an average rate of at least about 10° C. per minute under a pressure of at least 5.0 MPa, thereby forming the zeolite secondary structure.

4. A zeolite secondary structure obtained from zeolite primary particles comprising less than about 10% by weight of binders, wherein the secondary structure is obtained by a process comprising providing zeolite primary particles, heating the zeolite primary particles to above about 800° C. at an average rate of at least about 10° C. per minute under a pressure of at least 5.0 MPa, thereby forming the zeolite secondary structure.

5. The zeolite secondary structure according to claim 3, wherein the process comprises cooling at an average rate of at least about 1° C. per minute.

6. The zeolite secondary structure according to claim 3, wherein the maximum heating temperature is above about 800° C. up to about 1400° C.

7. The zeolite secondary structure according to claim 3, wherein the average heating rate is at least about 20° C. per minute.

8. The zeolite secondary structure according to claim 3, wherein the average temperature above about 800° C. is maintained less than about 60 minutes.

9. The zeolite secondary structure according to claim 1, wherein the zeolite primary particles are crystalline microporous aluminosilicate material.

10. The zeolite secondary structure according to claim 9, wherein the crystalline microporous aluminosilicate material has a framework density FD of up to about 21 T-atoms per 1000 Å³.

11. The zeolite secondary structure according to claim 1, wherein the zeolite primary particles have a crystallographic free diameter of the channels having most T-atoms ranging from about 0.3 nm up to about 1.3 nm.

12. The zeolite secondary structure according to claim 1, wherein the zeolite primary particles have an MFI framework type.

13. The zeolite secondary structure according to claim 1, wherein the zeolite primary particles have a pore size distribution with more than about 25% of the pore volume in pores with radii from about 10 to about 10000 nm.

14. Use of the zeolite secondary structure as defined by claim 1 as a catalyst.

15. The use of the zeolite secondary structure according to claim 1 in a process for the isomerisation of hydrocarbons.

16. The use of the zeolite according to claim 15, wherein xylene is isomerised.

17. A method for manufacturing a zeolite secondary structure, wherein the method comprises providing zeolite primary particles, heating the zeolite primary particles to above about 800° C. at an average rate of at least about 10° C. per minute under a pressure of at least 5.0 MPa, thereby forming the zeolite secondary structure.

18. A process for isomerisation of hydrocarbons comprising contacting a hydrocarbon feed with a zeolite secondary structure as defined by claim 1.

19. The process according to claim 18, wherein xylene is isomerised.
